(19) United States
(12) Patent Application Publication
(10) Pub. No.: US 2018/0071648 A1
(43) Pub. Date: Mar. 15, 2018

(54) System and Method for Operating Remote Controlled Toys Using Brainwaves

(71) Applicant: Sujit I. Chhatlani, Hung Hom (HK)

(72) Inventors: Sujit I. Chhatlani, Hung Hom (HK); Jayesh Sujit Chhatlani, Hung Hom (HK)

(73) Assignee: Sujit I. Chhatlani

(21) Appl. No.: 15/267,137

(22) Filed: Sep. 15, 2016

(51) Int. Cl. A63H 30/04; A61B 5/048; A63H 27/00; A63H 17/26

(52) U.S. Cl. CPC A63H 30/04; A63H 17/26; A63H 27/12; A61B 5/048

(57) ABSTRACT

A system and method for controlling a remote control assembly with precision using signals detected by low-cost brainwave sensors. A brainwave detection unit senses brainwaves and produces corresponding data signals. The brainwave detection unit is capable of detecting the intensity of alpha waves and beta waves without complex waveform analysis. A control unit receives the data signals from the sensors of the brainwave detection unit. The control unit contains circuitry that sets a range for the brainwaves. The range is divided into multiple levels. The control unit produces different control signals as the data signals rise and fall between the levels of the range. A remote controlled assembly is provided that operates in response to the different control signals.

### BACKGROUND OF THE INVENTION

### 1. Field of the Invention

In general, the present invention relates to systems and methods that convert brainwaves into electronic control signals. More particularly, the present invention relates to systems and methods that enable a remote controlled toy to be operated by a user's brainwaves.

### 2. Prior Art Description

Brainwaves are the common name given for neural oscillations that originate within the brain. Neural oscillations are the rhythmic or repetitive neural activity that occurs in the central nervous system. Neural tissue in the brain can generate oscillatory activity in many ways. The oscillatory activity can be driven either by mechanisms within individual neurons or by interactions between neurons. In individual neurons, oscillations can appear either as oscillations in membrane potential or as rhythmic patterns of action potentials, which then produce oscillatory activation of post-synaptic neurons. At the level of neural ensembles, synchronized activity of large numbers of neurons can give rise to macroscopic oscillations, which can be observed in an electroencephalogram or similar detection equipment positioned around sections of the head. Oscillatory activity in groups of neurons generally arises from feedback connections between the neurons that result in the synchronization of their firing patterns. The interaction between neurons can give rise to oscillations at different frequencies than the firing frequency of individual neurons. Two well-known examples of macroscopic neural oscillations are alpha wave activity and beta wave activity.

Alpha waves are neural oscillations in the frequency range of 7.5-12.5 Hz. Alpha waves arise from synchronous and coherent electrical activity of thalamic pacemaker cells within the brain. Alpha waves predominantly originate from the occipital lobe during wakeful relaxation with closed eyes. Alpha waves reduce significantly when a person opens his/her eyes or when a person relaxes thoughts, as in drowsiness and sleep.

Beta waves, or beta rhythms, are the terms used to designate the frequency range of human brain activity between 12.5 and 30 Hz (12.5 to 30 transitions or cycles per second). Beta waves are split into three sections: Low Beta Waves (12.5-16 Hz, “Beta 1 power”); Beta Waves (16.5-20 Hz, “Beta 2 power”); and High Beta Waves (20.5-28 Hz, “Beta 3 power”).

Low amplitude beta waves with multiple and varying frequencies are typically generated with active, busy, or anxious thinking and active concentration. Over the motor cortex, beta waves are associated with the muscle contractions that happen in isotonic movements and are suppressed prior to and during movement changes. Bursts of beta activity are associated with a strengthening of sensory feedback in static motor control and reduced when there is movement change.

Alpha waves and beta waves can be detected by passive sensors positioned around the head. Sophisticated sensors, such as those used during electroencephalography (EEG) or magnetoencephalography (MEG) can measure wave frequencies, waveforms and other nuances in the brain wave patterns. However, using far simpler and far less expensive detection circuits, a person can detect the intensity of alpha waves and beta waves and readily determine if a person is producing beta waves by concentrating or if a person is producing alpha waves by relaxing. It is also readily apparent, by detecting changes in the alpha waves, if a person's eyes are open or closed.

In the prior art, simple inexpensive circuits have been invented for passively detecting the intensity of brainwaves. Such circuits are exemplified by U.S. Pat. No. 3,896,790 to Dikman. Using low cost brainwave detection circuits, toy manufacturers have developed toys that are supposedly controlled by thoughts. Rather, such toys typically detect the intensity of alpha waves, which are proportional to a degree of relaxation, therein providing two command states. When the alpha waves are above a threshold, they constitute a first command. Conversely, when the alpha waves are below the threshold, they constitute a second command.

Having only two command states limits the application of prior art brain wave detection circuits to toys. Although two command states can be used to turn a toy on and off or turn a toy left and right, there is no ability to control a toy with precision. The results are toy systems that have simple on/off controls. Such simplified toys are exemplified by U.S. Pat. No. 8,157,609 to Hallian, entitled “Mind-Control Toys And Method Of Interaction Therewith”.

A need therefore exists for a toy system and an improved brain wave controller that can provide a toy with multiple commands, yet only requires the same low cost brainwave detection circuits utilized in the prior art. In this manner, multiple elements of a toy can be controlled with thoughts. Likewise, features of the toy can be provided with incremental controls, therein greatly enhancing the play value of the toy system. This need is met by the present invention as described and claimed below.

### SUMMARY OF THE INVENTION

The present invention is a system and method for controlling a remote control assembly with precision using signals detected by low-cost brainwave sensors. The system has a brainwave detection unit that senses brainwaves and produces corresponding data signals. The brainwave detection unit is capable of detecting the intensity of alpha waves and beta waves without complex waveform analysis.

A control unit is provided that receives the data signals from the sensors of the brainwave detection unit. The control unit contains circuitry that sets a range for the brainwaves. The range is divided into multiple levels. The control unit produces different control signals as the data signals rise and fall between the levels of the range.

A remote controlled assembly is provided that operates in response to the different control signals. The control signals are transmitted to the remote controlled assembly through a data link. By monitoring the intensity of alpha wave brainwaves and beta wave brainwaves, simple low cost circuitry can be used to determine if a person is attempting to relax, attempting to concentrate or is purposely winking. These conditions can be converted into incremental control signals that enable the remote controlled assembly to be operated with great precision.

### BRIEF DESCRIPTION OF THE DRAWINGS

For a better understanding of the present invention, reference is made to the following description of an exemplary embodiment thereof, considered in conjunction with the accompanying drawings, in which:

FIG. 1 is a view that shows the major components of the present invention system;

FIG. 2 is a schematic that illustrates an exemplary operational methodology for the present invention;

FIG. 3 shows the details of a scale response subroutine for use with alpha brainwaves;

FIG. 4 shows the details of a scale response subroutine for use with beta brainwaves; and

FIG. 5 shows a remote controlled assembly being operated by various control signal types.

### DETAILED DESCRIPTION OF THE DRAWINGS

Although the present invention system and method can be embodied in many ways, the embodiment illustrated shows the system being applied to specific remote control toys. The embodiment is selected in order to set forth one of the best modes contemplated for the invention. The illustrated embodiment, however, is merely exemplary and should not be considered a limitation when interpreting the scope of the appended claims.

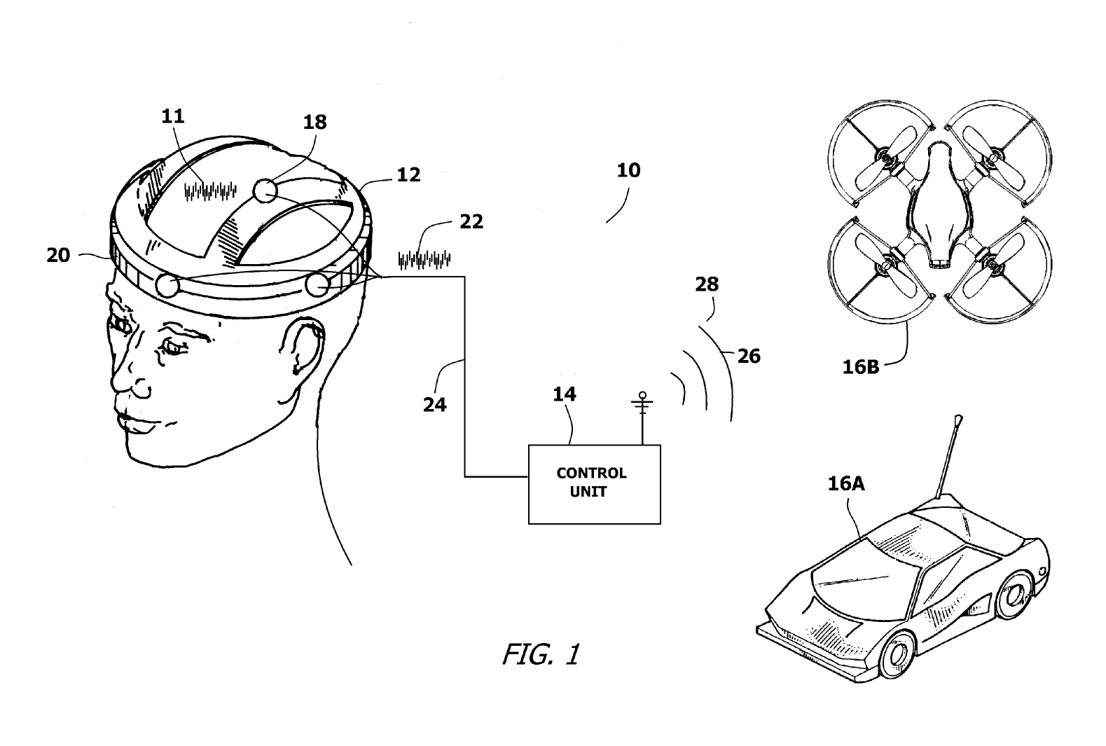

Referring to FIG. 1, the present invention system 10 is shown. The system 10 includes a brainwave detection unit 12, a control unit 14 and some form of a remote control toy 16A, 16B. The brainwave detection unit 12 contains sensors 18 that are capable of detecting brainwaves 11. The preferred brainwaves 11 that are being detected include both alpha waves and beta waves. The sensors 18 are positioned in a headpiece 20 that is worn about the head. Many brainwave detection units exist in the commercial marketplace. Many of these prior art brainwave detection units can be adapted for use as part of the present invention system 10.

The sensors 18 in the brainwave detection unit 12 detect the brainwaves 11 and convert those brainwaves 11 into a corresponding sensor data signal 22. The brain wave detection unit 12 communicates with the control unit 14 via a first data link 24. The first data link 24 can be a wire cable. However, wireless communication links, such as a BlueTooth® wireless link can also be used. The first data link 24 transmits the sensor data signals 22 to the control unit 14. The sensor data signals 22 are processed by the control unit 14 in a manner later described. The control unit 14 utilizes the sensor data signals 22 to generate control signals 26. The control signals 26 are transmitted from the control unit 14 to the remote control toy 16A, 16B using a second data link 28. The second data link 28 can be a wire cable. However, it is preferred that the second data link 28 be a wireless transmission, such as a radio transmission, an infrared light transmission or a laser light transmission.

The remote control toy 16A, 16B can be any type of toy that is operated by remote control. In the illustration, a remote control racecar and a remote control quadcopter 16B are shown by way of example. However, it will be understood that the remote control toy can be any rolling vehicle, any flying vehicle, any water vehicle, a robot, or any other assembly with articulating elements that can be remotely controlled.

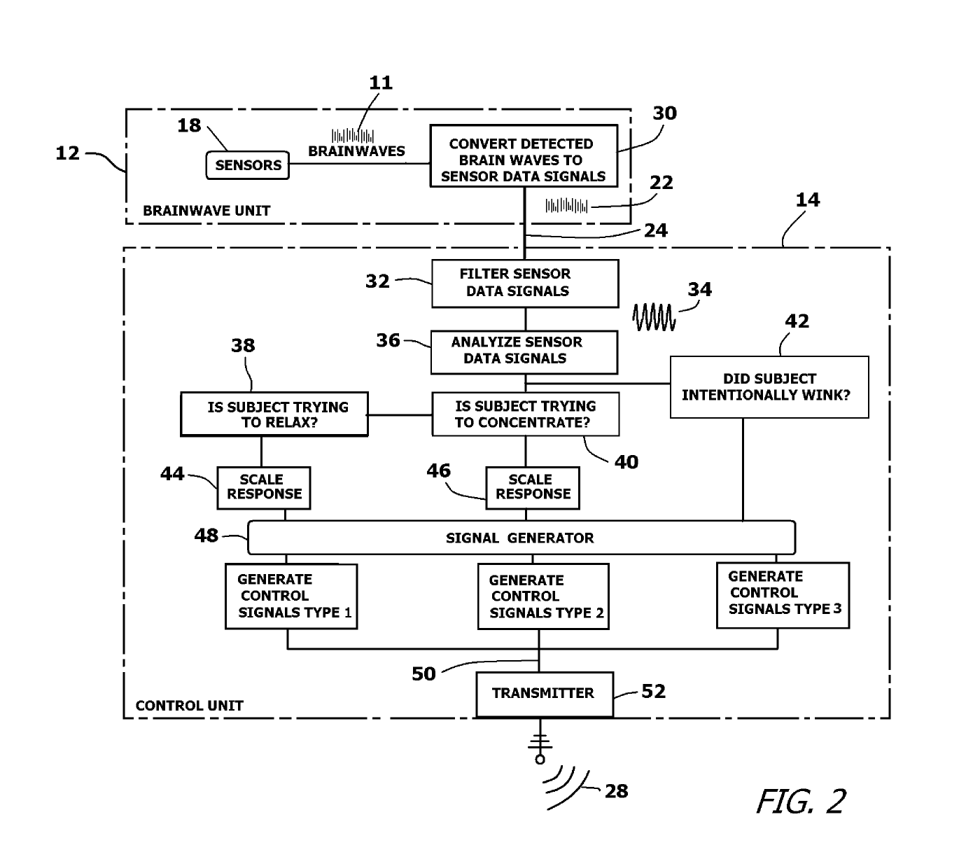

Referring to FIG. 2 in conjunction with FIG. 1, an operational overview of the system 10 is shown. As has been stated, within the brainwave detection unit 12 there are sensors 18 that detect brainwaves 11 and convert the detected brainwaves 11 into sensor data signals 22. See Block 30. Preferably, the brainwave detection unit 12 detects both alpha waves and beta waves.

The sensor data signals 22 are transmitted to the control unit 14 via the first data link 24. Within the control unit 14 is circuitry that processes the sensor data signals 22. As is indicated by Block 32, the sensor data signals 22 are first filtered to remove noise and features of the signal patterns that are unnecessary for analysis. This produces filtered sensor data signals 34. The filtered data signals 34 are then analyzed to detect progressive and abrupt changes in the intensity of the brainwave activity. Complicated analysis of brainwave waveforms is unnecessary. Rather, changes in alpha wave and beta wave intensity are monitored. This can be accomplished using inexpensive logic circuits and need not require a complex central processing unit.

As is indicated by Block 36, the filtered data signals 34 are analyzed. Alpha waves generally increase as a person relaxes. Beta waves generally increase as a person concentrates. The increase in alpha waves or beta waves is not sudden. Rather, it is progressive, wherein the waves increase in intensity depending upon the duration and effectiveness of the person's attempt to relax or concentrate. Conversely, alpha waves increase rapidly as a person closes his/her eyes. By detecting sudden increases in the intensity of the alpha waves, it can be determined that a person has closed his/her eyes. By measuring the duration of the event, it can be readily determined that a person has involuntarily blinked (duration of less than 0.2 seconds) or if a person has purposely winked (duration of over 0.5 seconds).

Using beta waves to monitor concentration levels, and alpha waves to monitor relaxation levels and winking events, a complex control system can be maintained. See Block 38, Block 40 and Block 42. Detected levels of concentration and/or relaxation are scaled between a maximum value and a minimum value. See Block 44 and Block 46. The maximum value and the minimum value can be preset but are preferably variables that are entered by a user during a set-up of the toy system 10. During the set-up of the toy system 10, a user can concentrate as hard as they can on some simple mental task, such as reciting the alphabet backwards while running in place. This concentration level can be set as the maximum concentration/minimum relaxation limit. Likewise, a user can be told to sit still and relax to a point just before they would fall asleep. This relaxation level can be set as the minimum concentration/maximum relaxation limit. The area between the two limits can be scaled, such as a scale from one to ten, or a scale from one to one-hundred. Points along the selected scale can be used as control events. The control events are used to generate control signals, as is later explained.

The control events are used as inputs to a signal generator 48 to create control signals 50. The control signals 50 are broadcast into the second data link 28. If the second data link 28 is wireless, the control signals 50 are broadcast by a transmitter 52.

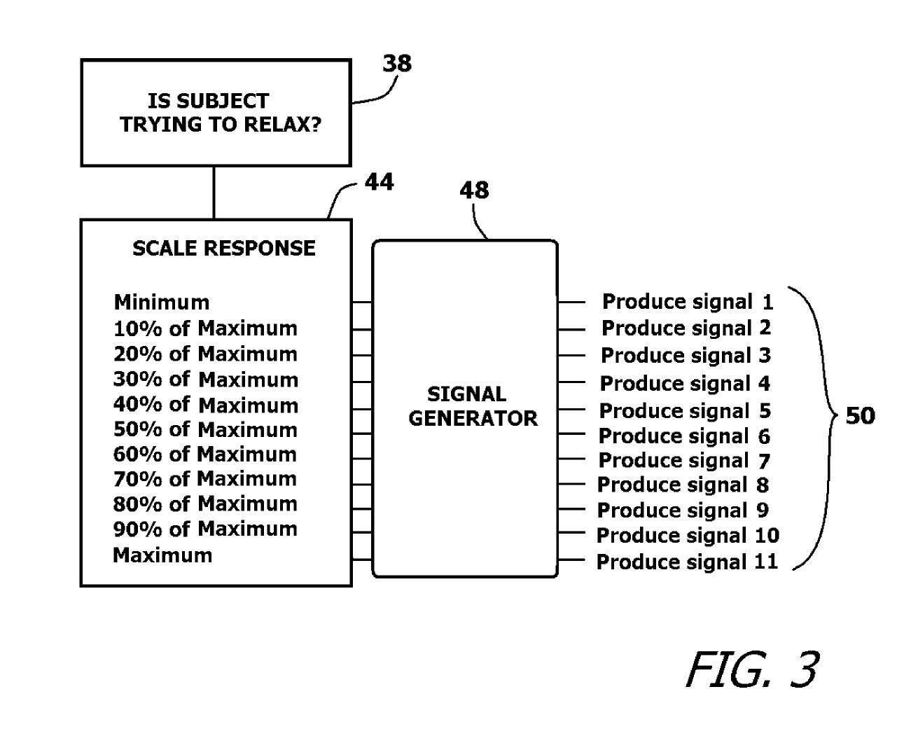

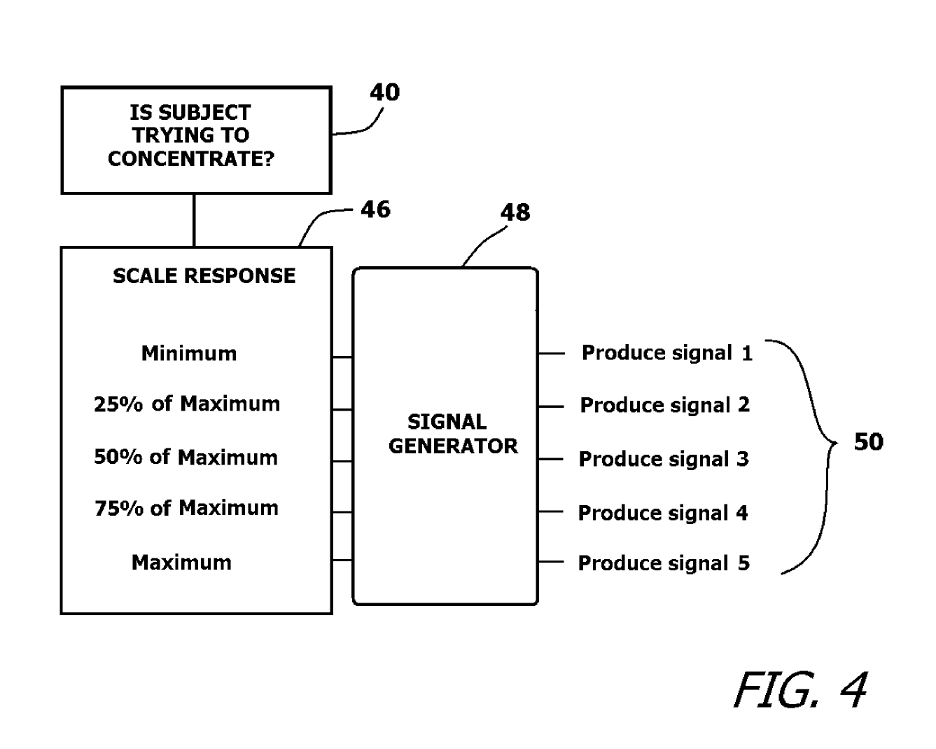

The scale responses to the levels of relaxation and concentration can be used to produce corresponding scaled control signals 50. The scale response for relaxation (Block 44 of FIG. 2) is detailed in FIG. 3. The scale response for concentration (Block 46 of FIG. 2) is detailed in FIG. 4. As is indicated in FIG. 3 and FIG. 4, a range is set between a minimum and a maximum value. The range is divided into levels. The number of levels corresponds to the number of signals that can be produced. Also the number of levels corresponds to the sensitivity of the response.

In FIG. 3, the scale response is divided into ten levels. As such, the scale response is capable of producing eleven signals. In FIG. 4, the scale response is divided into four levels. As such, the scale response is capable of producing five signals. The illustrated levels are merely exemplary and it will be understood that each scale response can be divided into any number of levels, depending upon the needs of the remote control toy 16A, 16B being operated.

It will therefore be understood that control signals are being created and broadcast in response to different brainwaves being detected by the brainwave detection unit 12. The control signals 50 being created are used to command and control the remote control toy 16A, 16B in a traditional manner. Most remote control toys require a minimum of throttle control signals and steering control signals to function properly. The throttle control signal controls the speed/engine power of the toy 16A, 16B. The steering control signals control the ability of the toy to turn left and right. Using throttle control signals and steering control signals, most remote control vehicles, boats, and drones can be controlled. However, to control the toy well, the throttle control signals and steering control signals need to incremental in nature. That is, the signals should be able to increase/decrease the throttle in small increments. Likewise, steering control signals used to turn the toy should also be in small increments. In this manner, small changes in speed and direction can be readily accomplished.

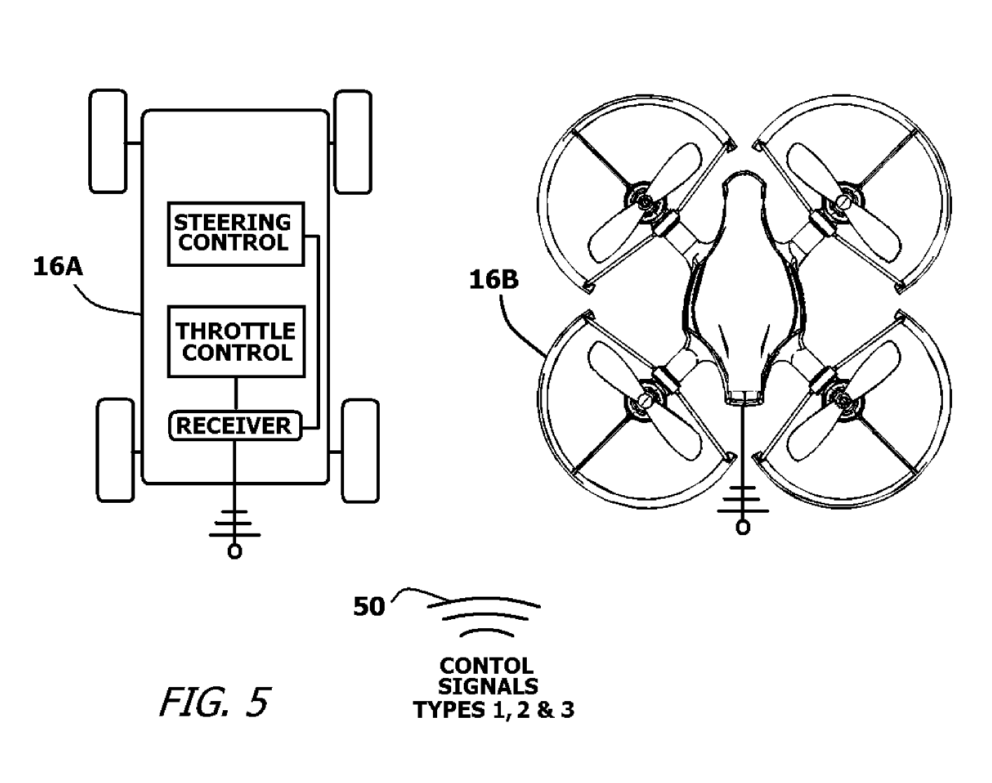

Referring to FIG. 5 in conjunction with FIG. 2, it can be seen that using the brainwave detection unit 12, brainwaves 11 can be detected that indicate if a person is attempting to concentrate or relax. The brainwave detection unit 12 can also detect if a person involuntarily winks. In the example illustrated, the winking command can be used to start and stop the remote control toy. The incremental signals associated with relaxation, see FIG. 3, can be used for steering. Likewise, the incremental signals associated with concentration, see FIG. 4, can be used for steering. Such control designations are merely an example. Many other control assignments can be used. For example, relaxation controls right turns and concentration controls left turns. Throttle can be controlled by winking. What is important is that multiple control signals can be generated by concentrating at different degrees. Likewise, multiple control signals can be generated by relaxing at different degrees. These control signals can be supplemented by signals generated by intentionally winking.

Using combinations of control signals 50 generated by relaxing, concentrating and winking, dozens of different command signals can be transmitted to the remote control toy. This enables a person to control the remote control toy with a high degree of precision. The result is that more sophisticated remote control toys can be utilized, therein increasing the play value of the system.

It will be understood that the embodiment of the present invention that is illustrated and described is merely exemplary and that a person skilled in the art can make many variations to that embodiment. For instance, the remote control toy can be any toy with a feature that is remotely controlled by a user. Likewise, the brainwave detection unit need not be a headset, but can be embodied into a hat or toy helmet. All such embodiments are intended to be included within the scope of the present invention as defined by the claims.

What is claimed is:

1. A system, comprising:
a brainwave detection unit that senses brainwaves and produces corresponding data signals;
a control unit that receives said data signals through a first data link, wherein said control unit sets a range with multiple levels for said data signals and produces different control signals as said data signals pass into said multiple levels of said range;
a remote controlled toy that operates in response to said different control signals; and
a second data link that transmits said control signals to said remote controlled toy from said control unit.

2. The system according to claim 1, wherein said brainwave detection unit senses both alpha wave brainwaves and beta wave brainwaves.

3. The system according to claim 1, wherein said first data link is a wireless link.

4. The system according to claim 1, wherein said second data link is a wireless link.

5. The system according to claim 2, wherein said control unit monitors said alpha wave brainwaves to determine if a wink longer than a threshold duration has occurred.

6. The system according to claim 5, wherein said control unit produces a first type of said different control signals should said wink occur.

7. The system according to claim 6, wherein said data signals contain data on alpha wave brainwaves, wherein said control unit monitors said alpha waves to determine intensity of said alpha wave brainwaves within said range.

8. The system according to claim 7, wherein said control unit produces a second type of said different control signals as said alpha wave brainwaves vary within said range.

9. The system according to claim 8, wherein said data signals contain data on beta wave brainwaves, wherein said control unit monitors said beta wave brainwaves to determine intensity of said beta wave brainwaves within said range.

10. The system according to claim 9, wherein said control unit produces a third type of said different control signals as said alpha wave brainwaves vary within said range.

11. The system according to claim 10, wherein said different control signals used by said remote controlled toy include said first type, said second type and said third type of said different control signals.

12. A system, comprising:
a headpiece that contains brainwave sensors for detecting brainwaves and producing corresponding data signals;
circuitry that receives said data signals and monitors said data signals across levels within a range, wherein said circuitry produces different control signals as said data signals pass between said levels of said range; and
a remote controlled assembly that operates in response to said different control signals.

13. The system according to claim 12, wherein said data signals are transmitted from said sensors to said circuitry through a first data link.

14. The system according to claim 13, wherein said control signals are transmitted from said circuitry to said remote controlled assembly through a second data link.

15. The system according to claim 12, wherein said sensors detect both alpha wave brainwaves and beta wave brainwaves.

16. The system according to claim 15, wherein said circuitry monitors said alpha wave brainwaves to determine if a wink longer than a threshold duration has occurred and produces a first type of said different control signals should said wink occur.

17. The system according to claim 15, wherein said circuitry monitors said alpha wave brainwaves to determine intensity of said alpha wave brainwaves within said range.

18. The system according to claim 17, wherein said circuitry produces a second type of said different control signals as said alpha wave brainwaves vary within said range.

19. The system according to claim 15, wherein said circuitry monitors said beta wave brainwaves to determine intensity of said beta wave brainwaves within said range.

20. The system according to claim 19, wherein said circuitry produces a third type of said different control signals as said beta wave brainwaves vary within said range.
